(19) United States
(12) Patent Application Publication
(10) Pub. No.: US 2008/0308111 A1
(43) Pub. Date: Dec. 18, 2008

(54) Finger shaped protection sleeve for inserting tampon

(76) Inventor: Jean-Paul Aronson, (US)

Correspondence Address: Aronson, Barentszstraat 3, Amsterdam, 1013NL, omitted

(21) Appl. No.: 11/764,192

(22) Filed: Jun. 16, 2007

(51) Int. Cl. A61F 5/37; B65D 43/00; B65D 83/00

(52) U.S. Cl. 128/846; 206/363; 229/124

(57) ABSTRACT

A finger fitting plastic disposable sleeve, giving the user's finger and nail protection while inserting a tampon, fitted in a small container holding multiple sleeves.

The invention relates to a finger fitting sleeve, with preference a disposable one made of low density polyethylene, which can be put around the finger before inserting a tampon with that finger, in order to prevent the finger and/or nail to come in direct contact with body fluids that cause the finger and/or nail to become unwantingly wet and/or smelling from these fluids.

The invention also relates to a little box (container) in which the sleeves are packed by more than 1 pieces, which among other properties has the possible property that it is as flat and small as possible to hold the selected amount of sleeves, and also that it has a size that makes it suitable for putting it in the pocket of trousers or other clothing. But this is not a must, because the box may have a bigger size and hold more sleeves, for example for keeping it at home or transporting it with one in a suitcase. Also it does not necessarily have to be a box. It can also be a sort of bag holder in which the sleeves are held, or any other shape and/or material which is suited for the task.

During insertion of a tampon the used finger and/or nail usually will get wet with body fluids. These can be body fluids that normally already exist in the vagina as well as body fluids that are created during the menstrual period. This results in a finger and/or nail touched by those fluids. Especially the female nail can be long, so fluids and consequently the smell will remain under it, even after washing the hands. It becomes even more annoying when the tampon has to be put in being on a location where it is more difficult or impossible to wash hands. The smell that is very persistent will exist for a long time, even after washing hands. The finger becomes temporary bearer of undesired matter and/or scents. Apart from matter and/or scent, it can be uncomfortable for many women to enter the vagina with a bear finger when inserting a tampon.

The invention aims to protect the finger and nail in such a way that problems arising during insertion of a tampon can be avoided.

According to the invention the solution can be reached by sliding a fitting sleeve over the finger and nail, before inserting a tampon.

With preference the sleeve, which can consist of 2 sides of finger shaped polyethylene or any other suitable material being put together thus creating a cylinder shaped finger sized sleeve resisting moist, has on the bottom side a short distance over which the 2 sides are not connected and so creating 2 loose ends with which the sleeve can be taken from the finger and/or be put on the finger. These loose ends can be any desired size best for serving this goal. Also it is possible that the sleeve does not have this loose ends, or it could have 1 instead of 2. The top of the sleeve has a round shaped edge, which ensures that there are no sharp corners or points going into the vagina. The sleeve is made in such a way that it easily slides over the finger. Also the material ensures that the aim of avoiding moist to get in contact with finger and/or nail is met, even when using it with a long nail. The material can be see through but does not have to be. It can be without colour or have a colour.

With preference the box is made out of carton, but this can also be any other material suitable for this purpose. It has a size which is as small as possible to ensure that there are as few obstakels as possible to carry it with one, but it can be bigger also for keeping it at home or in a suitcase. The box that should be small enough to carry it with one even in a pocket of trousers, should be just big enough to carry a certain amount of sleeves, for example 30 or 42 pieces. It is made in such a way that it is easy to be opened and reclosed. After opening the box the top sleeve, or any other sleeve, can be taken out. To prevent that after opening more than 1 sleeve comes out of the box, for example by wind or from any other cause, the sleevas with preference are fastened to the box. With preference in such a way that the method for fastening is not in sight, but it doesn't have to be like that, it could be in sight. The sleeves are fastened in such a way that it becomes easy to take out the sleeves one by one. This can be achieved by a perforated line between the part of the sleeves that stays in the box and the part of the sleeves that has to come out.

The invention will be presented in the following drawings.

Drawing number 1 shows a sleeve in not see through condition (A) put on one finger (D), almost to the point where the hand starts. It shows the 2 possible loose ends (B and C) for holding and pulling the sleeve in an easy manner, but this can also be just or none.

Drawing number 2 shows a see through sleeve (H) put on one finger (V). Here is shown that the sleeve is being put on the finger so far that the nail (F) and the phalanges (G) are easily covered by the sleeve. This is preferred but it is also possible to make the sleeve shorter to fit circumstances in which a shorter version is desired, for example during less existing body fluids.

Drawing number 3 shows a sleeve (K) with at the open end the 2 loose ends (J).

Drawing number 4 shows a carton being an unfolded box which when folding it at the cutting edges will form a closed box. The side shown as Al is the inside of the back side of the box. On that on the left side is fitted a piece of double sided tape (A3) which ensures that after assembling the box, the staple (A4) will stick to A3. Side A5 ensures that nore the staple, neither the tape is in sight. This is not a must but has preference for obvious aesthetic reasons. When the sleeves are fitted to the box by using the staple, side A4 can be fitted to the tape (A3). On the back of the side A5 that is visible, also a piece of double sided tape is put, so that the sides A3 and A4 and A5 can be folded together and held together and that after opening the box by the user no tape nor staple is visible.

To close the box or reclose it again, after taking out a sleeve for example, the sides A7 and A8 can be folded inwards first, followed by side A6, then followed by side A9 which can be slit into the opening A10 of side A9 which can be slit into the opening A10 of side A6. The sides A4 and All and A12 and A13 make sure that the box holds it's desired volume after folding it shut.

Drawing number 5 shows an angle view of the open (unfolded) box. With preference a staple that holds the sleeves together is also stapled through side V7, with the sleeves being located under that side V7, resting on side B4. B4 is in this drawing the inside of the back of the box. B3 are the two side flaps and B1 can be slit into the opening B5 of side B2 for closing the box again. In between sides V6 and V7 and in between the sides V7 and V8 the double sided pieces of tape can be put in order to keep all these sides situated/folded as shown in this drawing.

Drawing number 6 shows the box from the side, and containing some sleeves. The sleeves (D4) are held by a staple (D3) put through the non removable part (D6) of the sleeves and put through one side of the box. The perforated line (D5) connects the removable actual sleeves (D4) with the non removable part of every sleeve (D6). The layer that holds the staple is covered by the side under it and the side above it, all fitted together by using for example the double sided tape D1 and D2.

Drawing number 7 shows a sleeve (E3), as well as the by a perforation line attached non removable part (E1). The staple (E2) through that part of the sleeves has to ensure that a sleeve (E3) can be taken out of the box while the non removable part (E1) as well as all remaining sleeves, will stay put.

### DESCRIPTION

The invention relates to a finger fitting sleeve, with preference a disposable one made off low density polyethylene, which can be put around the finger before inserting a tampon with that finger, in order to prevent the finger and/or nail to come in direct contact with body fluids that cause the finger and/or nail to become unwantingly wet and/or smelling from these fluids.

The invention also relates to a little box (container) in which the sleeves are packed by more than 1 pieces, which among other properties has the possible property that it is as flat and small as possible to hold the selected amount of sleeves, and also that it has a size that makes it suitable for putting it in the pocket of trousers or other clothing. But this is not a must, because the box may have a bigger size and hold more sleeves, for example for keeping it at home or transporting it with one in a suitcase. Also it does not necessarily have to be a box. It can also be a sort of bag holder in which the sleeves are held, or any other shape and/or material which is suited for the task.

During insertion of a tampon the used finger and/or nail usually will get wet with body fluids. These can be body fluids that normally already exist in the vagina as well as body fluids that are created during the menstrual period. This results in a finger and/or nail touched by those fluids. Especially the female nail can be long, so fluids and consequently the smell will remain under it, even after washing the hands. It becomes even more annoying when the tampon has to be put in being on a location where it is more difficult or impossible to wash hands. The smell that is very persistent will exist for a long time, even after washing hands. The finger becomes temporary bearer of undesired matter and/or scents. Apart from matter and/or scent, it can be uncomfortable for many women to enter the vagina with a bare finger when inserting a tampon.

The invention aims to protect the finger and nail in such a way that problems arising during insertion of a tampon can be avoided.

According to the invention the solution can be reached by sliding a fitting sleeve over the finger and nail, before inserting a tampon.

With preference the sleeve, which can consist of 2 sides of finger shaped polyethylene or any other suitable material being put together thus creating a cylinder shaped finger sized sleeve resisting moist, has on the bottom side a short distance over which the 2 sides are not connected and so creating 2 loose ends with which the sleeve can be taken from the finger and/or be put on the finger. These loose ends can be any desired size best for serving this goal. Also it is possible that the sleeve does not have this loose ends, or it could have 1 instead of 2.

The top of the sleeve has a round shaped edge, which ensures that there are no sharp corners or points going into the vagina. The sleeve is made in such a way that it easily slides over the finger. Also the material ensures that the aim of avoiding moist to get in contact with finger and/or nail is met, even when using it with a long nail. The material can be see through but does not have to be. It can be without colour or have a colour.

With preference the box is made out of carton, but this can also be any other material suitable for this purpose. It has a size which is as small as possible to ensure that there are as few obstakels as possible to carry it with one, but it can be bigger also for keeping it at home or in a suitcase. The box that should be small enough to carry it with one even in a pocket of trousers, should be just big enough to carry a certain amount of sleeves, for example 30 or 42 pieces. It is made in such a way that it is easy to be opened and reclosed. After opening the box the top sleeve, or any other sleeve, can be taken out. To prevent that after opening more than 1 sleeve comes out of the box, for example by wind or from any other cause, the sleevas with preference are fastened to the box. With preference in such a way that the method for fastening is not in sight, but it doesn't have to be like that, it could be in sight. The sleeves are fastened in such a way that it becomes easy to take out the sleeves one by one. This can be achieved by a perforated line between the part of the sleeves that stays in the box and the part of the sleeves that has to come out.

The invention will be presented in the following figures.

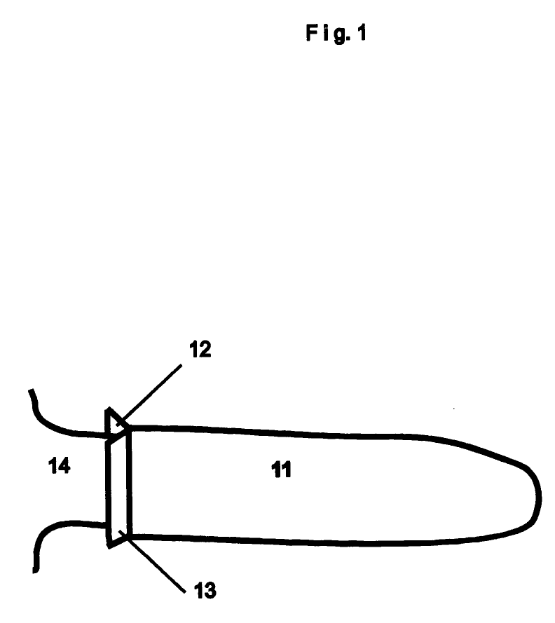

FIG. 1 shows a sleeve in not see through condition (11) put on one finger (14), almost to the point where the hand starts. It shows the 2 possible loose ends (12 and 13) for holding and pulling the sleeve in an easy manner, but this can also be just 1 or none.

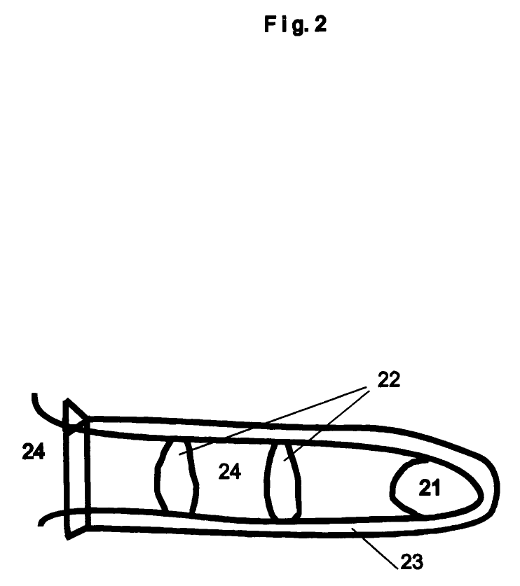

FIG. 2 shows a see through sleeve (23) put on one finger (24). Here is shown that the sleeve is being put on the finger so far that the nail (21) and the phalanges (22) are easily covered by the sleeve. This is preferred but it is also possible to make the sleeve shorter to fit circumstances in which a shorter version is desired, for example during less existing body fluids.

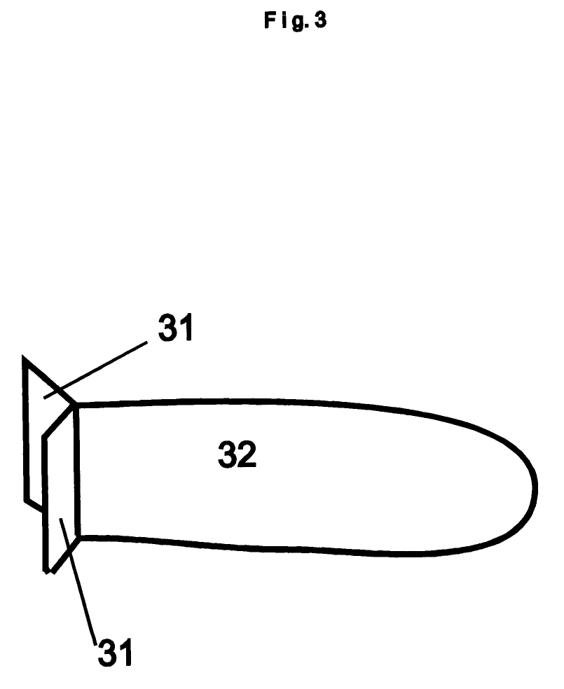

FIG. 3 shows a sleeve (32) with at the open end the 2 loose ends (31).

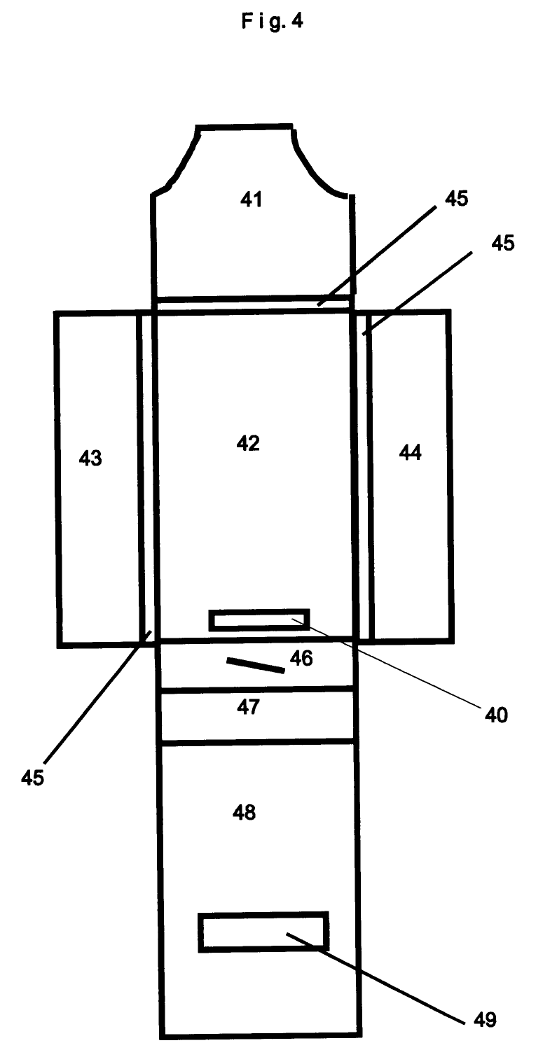

FIG. 4 shows a carton being an unfolded box which when folding it at the cutting edges will form a closed box. The side labeled nr. 42 is the inside of the back side of the box. On that on the left side is fitted a piece of double sided tape (40) which ensures that after assembling the box, the staple (46) will stick to (40). Side 47 ensures that nore the staple, neither the tape is in sight. This is not a must but has preference for obvious aesthetic reasons. When the sleeves are fitted to the box by using the staple, side 46 can be fitted to the tape (40). On the back of the side 47 that is visible, also a piece of double sided tape is put, so that the sides 40 and 46 can be folded together and held together and that after opening the box by the user no tape nor staple is visible.

To close the box or reclose it again, after taking out a sleeve for example, the sides 43 and 44 can be folded inwards first, followed by side 48, then followed by side 41 which can be slit into the opening (40) of side 48. The sides 46 and 45 make sure that the box holds it's desired volume after folding it shut.

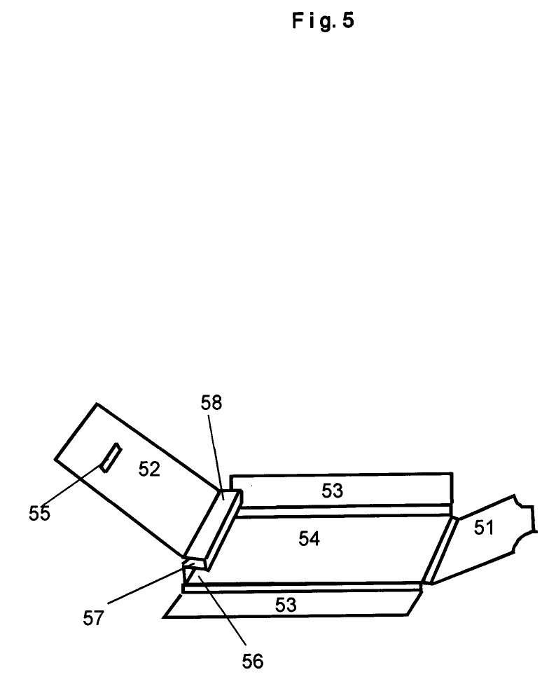

FIG. 5 shows an angle view of the open (unfolded) box.

With preference a staple that holds the sleeves together is also stapled through side 57, with the sleeves being located under that side 57, resting on side 54. Side 54 is in this drawing the inside of the back of the box. Sides 53 are the two side flaps and 51 can be slit into the opening 55 of side 52 for closing the box again. In between sides 56 and 57 and in between the sides 57 and 58 the double sided pieces of tape can be put in order to keep all these sides situated/folded as shown in this drawing.

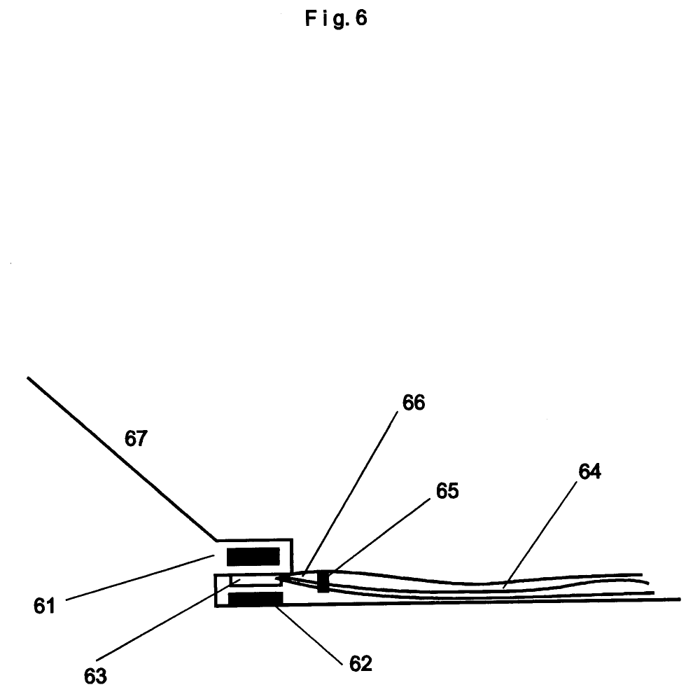

FIG. 6 shows the box from the side, and containing some sleeves. The sleeves (64) are held by a staple (63) put through the non removable part (66) of the sleeves and put through one side of the box. The perforated line (65) connects the removable actual sleeves (64) with the non removable part of every sleeve (66). The layer that holds the staple is covered by the side under it and the side above it, all fitted together by using for example the double sided tape 61 and 62.

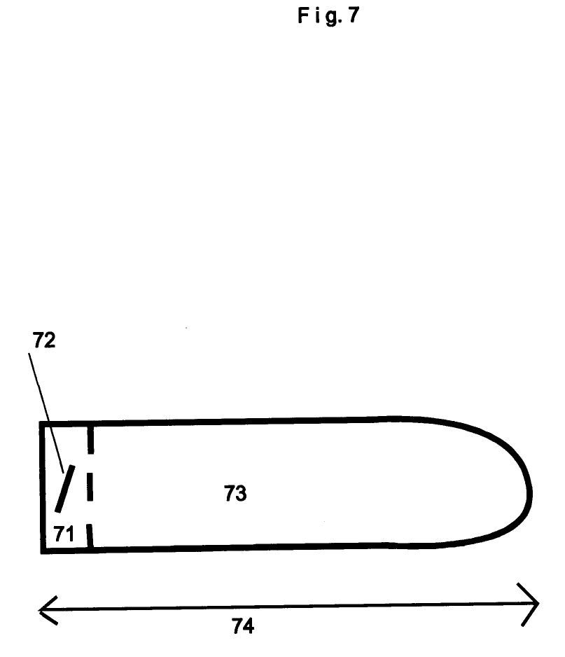

FIG. 7 shows a sleeve (73), as well as the by a perforation line attached non removable part (71). The staple (72) through that part of the sleeves has to ensure that a sleeve (73) can be taken out of the box while the non removable part (71) as well as all remaining sleeves will stay put.

The invention claimed is:

1. A disposable plastic sleeve in the shape of a finger, fitting around the finger by sliding it over the finger using the one open end of the sleeve, and so protecting that same finger and nail during insertion of a tampon by that same finger.

2. A disposable plastic sleeve according to claim 1, with at the open end of the sleeve, it having one or two loose flaps for holding and handling the sleeve.

3. A disposable plastic sleeve according to claim 1, protecting the finger and or nail against any kind of moist and or fluids and/or any other substances that a person wants to prevent of coming in to direct contact with that finger and or nail.

4. A disposable plastic sleeve according to claim 1, consisting of 2 layers of plastic fitted together with the edges, leaving open the straight end or top, and so forming a sleeve that fits a finger.

5. A carton box that holds multiple plastic sleeves as described in claim 1, which has the minimum required size for holding the amount of sleeves that it does, and so being of a size that fits into a handbag or a pocket of trousers, jackets or any other clothes.

6. A carton box according to claim 6, of which one side is for easy opening and reclosing of the box, and so making it possible to take out the required sleeve or sleeves without damaging the box.
